(19) United States
(12) Patent Application Publication
(10) Pub. No.: US 2009/0124973 A1
(43) Pub. Date: May 14, 2009

(54) Insertion mechanism for use with a syringe

(76) Inventors: Eduardo D'Agostino, Bedford, MA (US); Karin Maback, Kingston, NH (US)

Correspondence Address: LAMBERT & ASSOCIATES, P.L.L.C., 92 STATE STREET, BOSTON, MA, 02109-2004, US

(21) Appl. No.: 12/214,160

(22) Filed: Jun. 18, 2008

Related U.S. Application Data
(60) Provisional application No. 61/002,722, filed on Nov. 9, 2007.

(51) Int. Cl. A61M 5/46

(52) U.S. Cl. 604/117

(57) ABSTRACT

A needle preferably for the delivery of ophthalmic regional anesthesia, wherein the needle includes a hub and a shaft having a plurality of markings to indicate the depth of the needle after insertion into an individual during a medical procedure, thereby enabling a practitioner to gauge the exact measurement of the needle at all times during the operation.

### CROSS REFERENCE TO RELATED APPLICATION

This application claims the benefit of prior U.S. Provisional Application No. 61/002,722 filed on Nov. 7, 2007, the contents of which are incorporated herein by reference.

### BACKGROUND OF THE INVENTION

1. Field of the Invention

The instant invention relates generally to needles and in particular to a hypodermic needle for use with a syringe, preferably to assist physicians in the delivery of ophthalmic anesthesia.

2. Description of the Related Art

There are approximately 2.7 million Cataract surgeries performed annually in the United States each year, in addition to approximately 40,000 Corneal Transplants, Glaucoma and Retina surgeries. According to recent data, around 60% of the above-mentioned surgeries are performed using an ophthalmic regional block, peribulbar or retrobulbar as the method of delivering anesthesia to a patient. One of the primary reasons for this approach is that for any surgery to be successful, a surgeon requires absolute control over the procedure. As such, many surgeons are moving away from the unpredictable patient movements associated with topical anesthesia and toward the enhanced control offered through peribulbar or retrobulbar blocks.

These ophthalmic anesthesia regional blocks always employ the use of a hypodermic needle and the injection of a local anesthetic drug around and behind the eye. There are several techniques for performing this procedure; however most involve the needle being introduced into the orbital cavity between the eye and the orbital wall, wherein the needle always is tangentially oriented with the eye. Furthermore, there are predominantly three sites of insertion of the needle during these procedures (see FIG. 1):
1) Inferior-temporal: The needle advances between the eye and the inferior-external wall of the orbital cavity;
2) Medial: The needle advances between the eye and the medial wall of the orbital cavity;
3) Superior-temporal: The needle advances between the eye and the superior-external wall of the orbital cavity.

During the performance of these anesthesia regional block techniques, the direction of the needle and the angle of insertion are adjusted at least twice at predetermined needle depths, which is directly dependent on the axial length of eye (on average approximately 23.5 mm). Therefore, the correct depth and direction of the needle tip in regards to the axial length and equatorial plane of the eye, along with other vital structures are of great importance in order to avoid complications.

Several types of complications may arise during these procedures, including but not limited to globe penetration and perforation, venous and arterial hemorrhage, optic nerve damage, along with nerve and muscle injuries. In general, the problems associated with these procedures relates to a patient's safety, in that current needles for the delivery of anesthesia do not provide a precise method to avoid these risks. Furthermore, all of the present solutions relate to a practitioner's experience and knowledge of the involved region of the eye to reduce complications, rather than providing more accurate instruments. Moreover, all current solutions for the reduction of complications are based in theory, focusing on knowledge of the anatomy involved (i.e. eye and orbital region), and on the imaginary/subjective appreciation of the depth and orientation of the needle. Thus, once the needle is inserted into the tissue between the eye and the orbital cavity, the exact/objective recognition of the needle depth and orientation is lost, and the practitioner must solely rely on their experience and knowledge of the given anatomy.

The procedure in more detail comprises changes in the needle direction and angle of insertion, wherein the adjustments made depend mainly on the axial length (i.e. anterior-posterior diameter) of the eye; the correct depth, direction and orientation of the needle tip in relation to both the axial length and the equatorial plane (i.e. half of the anterior-posterior diameter of the eye) to avoid serious complications.

Presently, every needle available for ophthamalic regional blocks (i.e. peribulbar/retrobulbar) lacks any indication of the needle's depth and orientation (i.e. right, left, upper or lower) of the distal end (i.e. tip) once the needle has been inserted into the orbital cavity. Thus, present needles do not offer or provide an exact and objective approach to accurately perform this procedure. For example, after insertion of the needle into the orbital cavity, it is extremely difficult to calculate whether the needle has reached a depth of 6.25 mm or 12.5 mm, in determining when to adjust the needle and angle of insertion. (See FIG. 2—illustration of change of needle direction during procedure)

Furthermore, it is extremely difficult for any practitioner to know exactly when half the needle (whether it be a 25 mm or 32 mm needle) is at the level of the anterior edge of the cornea (i.e. the most anterior bulged coat of the eye), at the level of the iris (i.e. the circumferential color structure surrounding the pupil), or at the level of the equator/equatorial plane). As discussed above, knowledge of the relationship between the depth of the needle and the anatomical landmarks are essential in making the necessary corrections (i.e. medial and/or upper) on the direction of the needle pathway. Lastly, current data suggests that when advancing the needle during insertion, the bevel of the needle should be facing the eye globe. Needles that are presently available for ophthalmic anesthesia do not provide any indication to the practitioner of the orientation of the bevel and tip of the needle, once the needle has entered the orbital cavity.

The instant invention relates to a needle, preferably for use in the delivery of ophthalmic anesthesia that includes several markings equidistantly spaced along the shaft of the needle to assist a practitioner in determining the orientation and depth of the needle, preferably during medical procedures. Many devices have been developed to provide some type of assistance to practitioners engaged in medical procedures using a needle, preferably for the delivery of ophthalmic anesthesia; however no device currently provides both the depth and orientation of the needle during the procedure.

Ultimately, it is the design goal for such a needle to indicate the depth and orientation of the needle for assistance during medical procedures, wherein the needle includes a plurality of markings along the shaft of the needle. A large number of needles are known in the art, and in fact are in wide use in the industry. But there exists in the art no needle that includes a plurality of markings to assist a practitioner in visually determining the depth and orientation of the needle during a medical procedure; in summary, investigation of these disclosed devices illustrates that presently, there is no single device known in the art or combination thereof that meets the requirement of a needle having a plurality of markings that enable a practitioner to determine the depth and orientation of the needle during use, preferably for medical procedures related to ophthalmic regional anesthesia.

### SUMMARY OF THE INVENTION

The instant invention, as described further herein, imparts a novel insertion mechanism, preferably a needle, which encompasses the advantages of other needles, but allows for a practitioner to ascertain the depth and orientation of the needle after insertion, preferably during a medical procedure, by visual inspection of a plurality of marks locatable on the shaft of the needle. The instant invention as illustrated herein, is clearly not anticipated, rendered obvious, or even present in any of the prior art mechanisms, either alone or in any combination thereof.

The primary object of the instant invention is to provide a needle, preferably for the delivery of ophthalmic anesthesia, capable of providing a practitioner the depth and orientation of the needle after insertion.

Another object of the instant invention is to provide a needle that improves a patient's safety during ophthalmic regional anesthesia, wherein the needle includes a plurality of markings along the shaft to indicate both depth and orientation to the practitioner.

Another object of the instant invention is to provide a needle that is easy and simple to operate for a practitioner during performance of ophthalmic regional anesthesia.

Another object of the instant invention is to provide a needle, preferably for the delivery of ophthalmic anesthesia having a plurality of markings on the shaft that is cost-effective to manufacture and produce.

Another object of the instant invention is to provide a marked needle, having a plurality of markings, preferably for use by a practitioner to assist during operation of regional block anesthesia in adjusting the needle direction and angle of insertion based on a patient's axial length of the eye.

Another object of the instant invention is to provide a needle that includes a plurality of markings to assist teaching hospitals where ophthalmic blocks are taught, so that the instructor is able to gauge the depth and orientation of the needle during use by a student.

There has thus been outlined, rather broadly, the more important features of the marked needle in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Advantages of the present invention will be apparent from the following detailed description of exemplary embodiments thereof, which description should be considered in conjunction with the accompanying drawings, in which:

FIG. 1 is a diagrammatic perspective view of the various areas of insertion of a needle into a patient's eye during delivery of ophthalmic anesthesia.

FIG. 2 is a diagrammatic perspective cut-away view of the orbital region of the eye, illustrating the various positions of a needle during performance of a retrobulbar block, specifically demonstrating the angle and orientation change of the needle from initial to final insertion.

FIG. 3 is a diagrammatic perspective cut-away view of the orbital region of the eye, demonstrating the various measurements necessary to ascertain prior to commencement of a regional block.

FIGS. 4A-D illustrates a diagrammatic perspective side view of the orbital region, in relation to position of a prior art needle when performing a retrobulbar block using Harvey's Technique.

FIGS. 5A-C illustrates a diagrammatic perspective side view of the orbital region, in relation to position of a prior art needle when performing a retrobulbar block using Hamilton's Technique.

FIG. 6 illustrates a diagrammatic perspective seen from a view (transversal plane) above the orbital region, in relation to position of the instant invention when performing a Medial Compartment Peribulbar Block on the left eye.

FIG. 7 illustrates a diagrammatic perspective side view of the orbital region, in relation to position of the instant invention when performing a Superotemporal Peribulbar Block.

FIG. 8 illustrates a diagrammatic perspective view of a prior art needle.

FIG. 9 illustrates a diagrammatic perspective view of the instant invention, wherein the needle includes a hub and a shaft, the shaft having a plurality of markings to indicate depth of the needle after insertion during a medical procedure.

FIG. 10 illustrates a diagrammatic perspective view of the instant invention, wherein the hub includes a linear demarcation to indicate orientation of the bevel of the needle after insertion.

FIGS. 11A-D illustrates a diagrammatic perspective side view of the orbital region, in relation to position of the instant invention when performing a retrobulbar block using Harvey's Technique using the measurements contained in Example 1.

FIGS. 12A-D illustrates a diagrammatic perspective side view of the orbital region, in relation to position of the instant invention when performing a retrobulbar block using Harvey's Technique using the measurements contained in Example 2.

FIGS. 13A-D illustrates a diagrammatic perspective side view of the orbital region, in relation to position of the instant invention when performing a retrobulbar block using Harvey's Technique using the measurements contained in Example 3.

FIGS. 14A-B illustrates a diagrammatic perspective side view of the orbital region, in relation to position of the instant invention when performing an inferotemporal retrobulbar block using Hamilton's Technique using the measurements contained in Example 4.

FIG. 15 illustrates a diagrammatic perspective seen from above (transversal plane) view of the orbital region, in relation to position of the instant invention when performing a medial compartment block of the left eye using the measurements contained in Example 5.

FIG. 16 illustrates a diagrammatic perspective side view of the orbital region, in relation to position of the instant invention when performing a superotemporal peribulbar block using the measurements contained in Example 3.

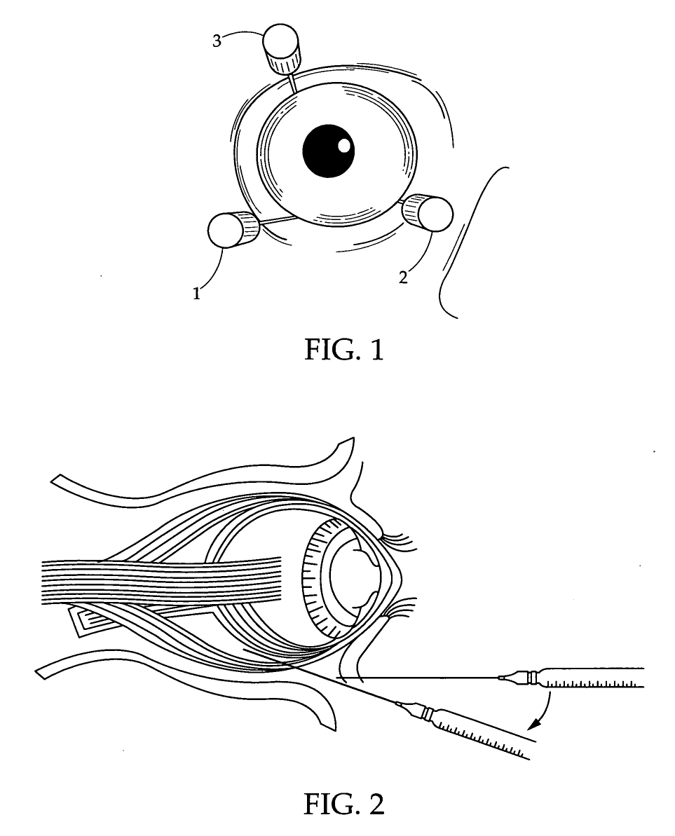

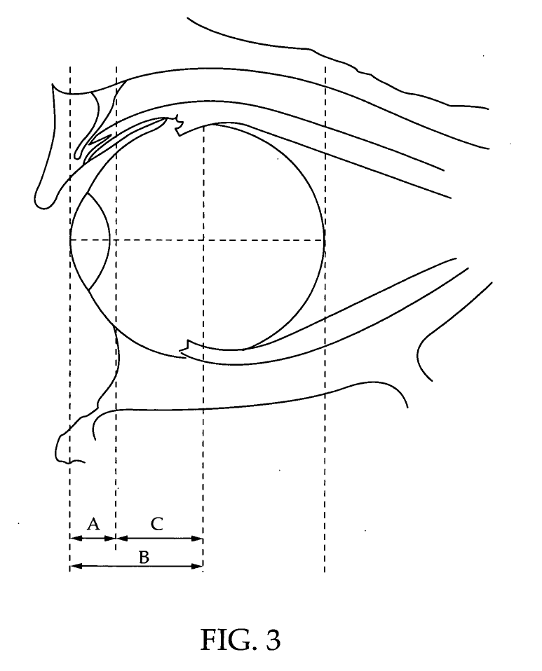

### DETAILED DESCRIPTION OF THE PREFERRED EMBODIMENT

Prior to setting forth the invention, it may be helpful to an understanding thereof to set forth definitions of certain terms to be used hereinafter.
Orbital Cavity: The bony cavity in the skull containing the eyeball and its associated muscles, vessels and nerves; also known as the eye socket.
Orbital Wall: The bony structure forming the orbital cavity; the four walls surrounding the cavity include the superior, inferior, medial (i.e. nasal) and the lateral (i.e. temporal).
Globe: The eye proper without the appendages; also known as the eyeball.
Axial Length: The anterior-posterior internal length of the globe (i.e. eyeball), wherein one half of the axial length is equal to the length of the equatorial plane.
Equatorial Plane: The imaginary line/plane located at the level of half of the distance from the anterior to posterior pole of the eye (i.e. widest plane of the eye globe).
Sclera: The most outer, semi-rigid (white color) coat of the eye. Maintains the shape of the eye and continues anteriorly with the cornea.
Cornea: The transparent bulging front part of the eye.
Cornea-Sclera Junction: It is the point at which the sclera (white) continues with the cornea (transparent) and is marked by a grey line called Limbus. The lateral (external) and medial (internal) junction/limbus are two essential anatomic landmarks for ophthalmic regional blocks.
Iris: The sphincter around the pupil of the eye; it functions as a muscular diaphragm controlling the amount of light entering the eye by varying the diameter of its aperture (i.e. the pupil).
Inferior Orbital Rim: The Lower, most anterior edge of the orbital cavity.
Saggital Plane: An imaginary plane that travels from the top to the bottom of the body, dividing it into left and right portions (i.e. it divides the eye into a right and left half).
Transversal Plane: An imaginary plane that divides the body into superior and inferior parts.
Lower Temporal Orbital Rim: The lowest, most lateral edge of the orbital cavity.

Prior to performance of this procedure, it is necessary to have two specific measurements in order to asses the needle/globe relationship:

The axial length (“AL”) as described above constitutes the anterior-posterior internal length of the eye globe. Thus, when passing a needle under the globe, the equatorial plane (widest plane of the eye globe) is the point where the needle can be safely turned upwards aiming for an endpoint behind the eye.

The other measurement which must be ascertained prior to the commencement of the operation is the distance from the cornea to the infraorbital rim (“IR”), which constitutes the initial insertion point of the needle (see FIG. 3). In one embodiment, the cornea can be right above the infraorbital rim, whereas alternatively, the cornea may reside in front of the rim on a “protruding” eye. As such, this measurement can easily be estimated by using a simple measuring stick and observing the cornea/orbital rim relationship from the side of the patient.

FIG. 3 illustrates the various measurements involved during operation of the above-referenced technique. Specifically, distance A refers to the distance from the cornea to the insertion point at the orbital rim (i.e. infraorbital rim). Distance B measures the distance from the cornea to the equator, which is equal to approximately half of the axial length. Furthermore, distance C specifies the distance the tip of the needle will be advanced before reaching the equator; distance C is calculated as distance B minus distance A.

Furthermore, it may be helpful to an understanding thereof, definitions of certain medical procedures and techniques to be used herein.

### Current Techniques

Current needles are sharp or blunt, with beveled tips. A beveled edge refers to an edge that is not perpendicular to the face of the needle. Most practitioners consider it safer to have the bevel of the needle face the globe as the needle approaches the equator and passes beyond it. If the angle of the bevel is such that the heel of the bevel opening will touch the surface before the tip, the tip will tend not to dig in. Herein will be described four of the most common currently used techniques, using an unmarked needle, preferably for the delivery of ophthalmic anesthesia, using the only needle presently available. After a description of these techniques, the instant invention will be disclosed along with a description of the below techniques utilizing the instant invention.

### Retobulbar Block

### Inferotemporal Retrobulbar Block—Harvey's Technique

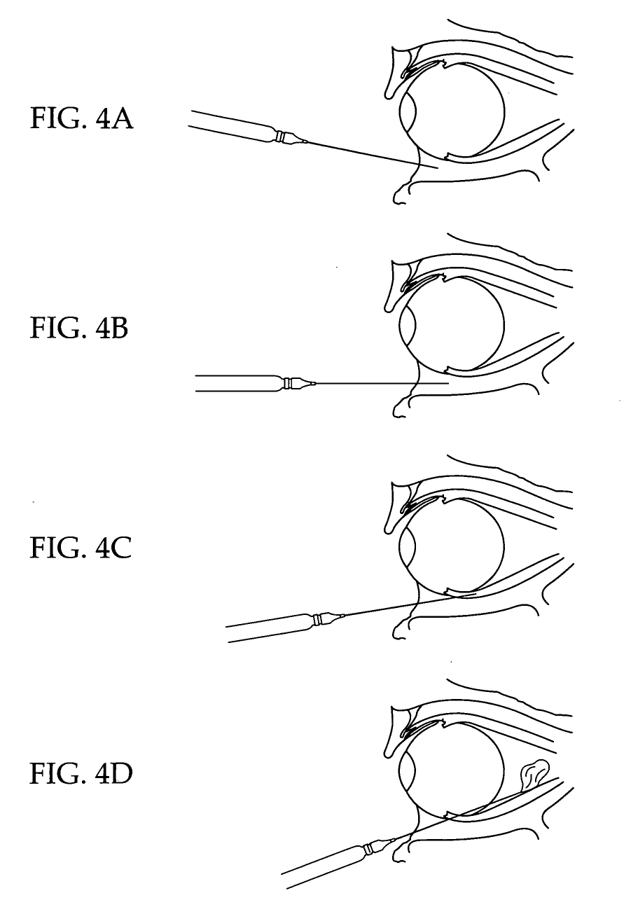

Harvey's technique is based on exact mathematical calculations; however distances A-C described above and illustrated in FIG. 3 are estimated during the advancement of needle through the orbital cavity using presently available needles. FIGS. 4A-D illustrates the method and procedure for accurately performing a retrobulbar block using Harvey's Technique. In FIG. 4A, the insertion of a needle preferably occurs, two millimeters inferior to the globe, wherein the bevel of the needle is oriented towards the globe at an angle of one hundred twenty degrees to the orbital floor. FIG. 4B illustrates the point of the procedure wherein redirection of the tip of the needle occurs, thereby orienting the needle parallel with the visual axis of the patient's eye. After redirection, the practitioner continues with slow advancement of the needle posteriorly, while simultaneously estimating the distance to the equatorial plane, and eventually passing the equatorial plane of the globe with the tip of the needle. FIG. 4C illustrates where a second adjustment of the needle occurs, specifically wherein the tip of the needle angles upwards. During this time, the practitioner continues with a slow advancement of the needle behind the globe to a depth of twenty-five millimeters. FIG. 4D illustrates when the needle has reached the desired depth behind the globe, thereby enabling the practitioner to inject the predetermined amount of anesthesia behind the eye.

### Inferotemporal Retrobulbar Block—Hamilton's Technique

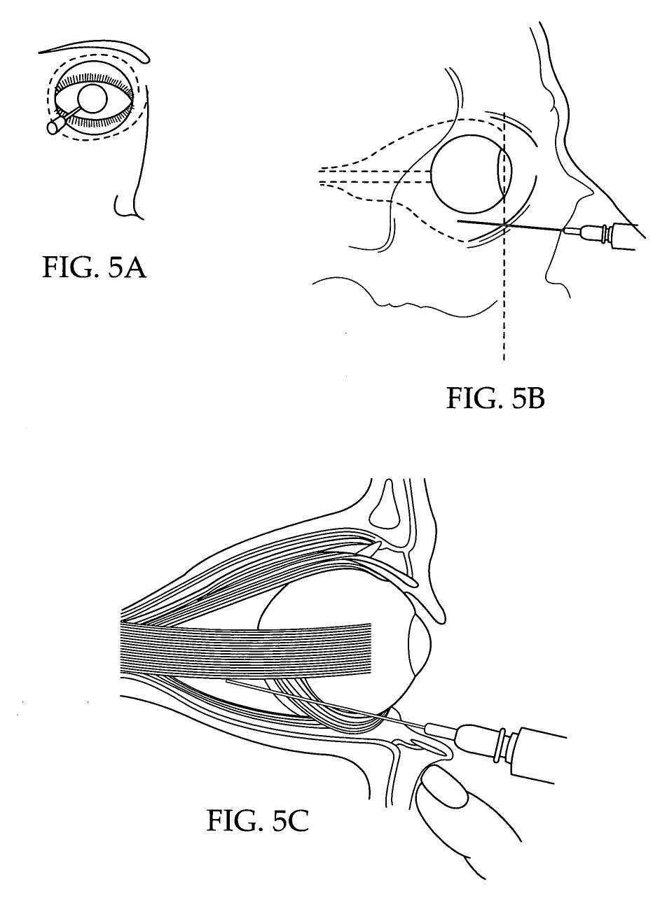

During this procedure, the tip of the needle enters the orbital cavity (either transconjunctival or transcutaneous route) at the lower temporal orbit rim (as seen in FIG. 3), which is slightly up from the orbital floor and very close to the bone. At this point, the distal half (i.e. leading edge) of the needle is advanced along the sagittal plane and oriented parallel to the orbital floor, until half of the needle has reached the plane of the iris (see FIGS. 5A-5C), thereby indicating that the tip of the needle has passed the equatorial plane of the globe. Moving forward with the procedure, the bevel of the needle should be oriented towards the globe, thereby keeping the tip of the needle away from the sclera and avoiding possible complications, such as a perforation of the globe.

A variation of the technique used by Dr. Gary Fanning, author of one of our references book, maintains the bevel of the needle facing the eye of the globe during insertion of the needle until the tip of the needle is well beyond the equatorial plane of the globe. In this embodiment, once the tip of the needle passes by the equatorial plane, the needle is adjusted one hundred eighty degrees, thereby guiding the tip of the needle toward the retrobulbar space and away from the orbital wall. At this point, the tip of the needle is oriented medially and slightly upward, thus enabling the practitioner to aim for an imaginary point behind the globe, wherein the axis is formed by the pupil and macula (i.e. the posterior center/pole of the EYE—See FIGS. 5A-C). It is important to note, that during performance of this technique, the needle must be inserted until the area where the needle and the hub are joined together, is at the level of the iris. However recently, most practitioners recommend a more cautious depth of orbital insertion, namely being until the needle is one inch inside of the orbital cavity meaning that an imaginary one inch mark of the needle should be at the level of the iris.

FIG. 5A as described above, illustrates initial insertion of the tip of the needle at the lower temporal orbital rim, which is positioned slightly upward from the orbital floor and in close proximity to the bone. FIG. 5B illustrates when approximately half of the needle reaches the plane of the iris, thus indicating the tip of the needle has passed the equatorial plane of the globe. Then, as described above, the tip of the needle is turned medially and slightly upward, thus aiming for a final position behind the globe, where the local anesthetic will be injected (see FIG. 5A). Lastly, FIG. 5C illustrates the final position of the needle behind the globe, wherein, the imaginary line, as referenced above has been reached, thereby enabling injection of the anesthesia by a practitioner.

### Peribulbar Block—Medial Compartment Peribulbar Block

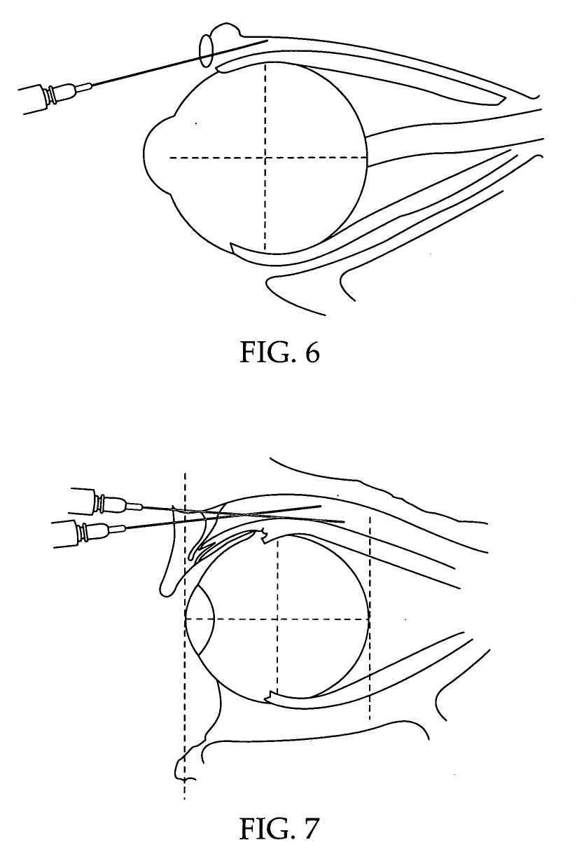

During this operation, the area of insertion and in focus is the medial canthal area (i.e. internal/nasal angle of the orbit—See FIG. 6). Prior to insertion, the bevel of the needle will be facing the globe to minimize any possible the risk of perforation of the globe. Once the orientation of the needle is correct, the tip of the needle will be inserted transconjunctivally into the depression next to canthal fold.

At this point, the needle should be oriented towards the medial orbital wall and advanced carefully until the needle comes into initial contact with the bone of the wall; it is important to note that this bone is extremely thin so the practitioner must be especially cautious during this movement. Once the wall has been contacted, the needle is withdrawn at a distance of one to two millimeters and reoriented to be subsequently inserted parallel to both the orbital wall and the orbital floor. During this part of the procedure, the needle will be inserted with a five degree medial angle to avoid any complications with the medial rectus muscle. The practitioner must understand that the needle should not be inserted more than one inch (i.e. until the hub/needle junction in a twenty five millimeter needle reaches the level of the iris—or utilizing a thirty two millimeter needle, when an imaginary one inch mark has been reached). There is recent literature that suggests there is no need to insert the needle more than eighteen and three quarter's millimeters. It should be noted in this case that the practitioner performing this procedure must use an imaginary mark of eighteen and three quarter's millimeters on the needle to evaluate exactly when to cease insertion of the needle.

A modification of this procedure centers on some practitioners thinking that it is preferable to have the bevel opening facing the nasal side of the orbital cavity instead of facing the globe. Regardless of the exact technique utilized with respect to the position of the bevel opening, it is important to realize that once the tip of the needle has been inserted, the exact orientation of the bevel opening must be ascertained.

FIG. 6 illustrates the above-described procedure in more detail, specifically when the bevel of the needle is oriented towards the globe; the practitioner inserts the needle with at a five degree medial angle. Once the needle has been inserted, the practitioner proceeds medially and posteriorly to a depth of eighteen and three quarter's millimeters, or twenty five millimeters according to the technique of preference. After negative aspiration occurs, the practitioner may inject three milliliters of the anesthesia solution. Lastly, the needle is withdrawn with the bevel facing the globe to avoid the tip of the needle pointing towards the eye globe.

### Peribulbar Block—Superotemporal Peribulbar Block

This procedure is undertaken in the instance when patients in who after the above-described methods were concluded, still possess a strong activity in the superior rectus and levator palpbrae superioris muscles (i.e. meaning the ability to open the eye lid by the patient), In this instance, a superotemporal extraconal injection of local anesthetic is used. Insertion of the needle occurs through the skin of the superior lid, approximately three millimeter lateral to the sagittal plane (see FIG. 7) of the lateral limbus (corneo-scleral junction) at level of the superior orbital rim and is oriented upwardly towards the roof of the orbital cavity, preferably with a medial component of approximately five degrees. Thus, the tip of the needle “walks” along the bone of the orbital roof in a curvilinear fashion, with the bevel opening of the needle oriented towards the globe, thereby diminishing the risk of perforation; once the needle reaches a depth of twenty five millimeters, the practitioners makes the injection of anesthesia. An alternative to this approach, as with the medial compartment block described above, some practitioners prefer the bevel opening facing the orbital roof while performing the superotemporal injection. Again it must be remembered, regardless of the technique used as the position of bevel opening is concerned, it is crucial to know once the needle is inserted, the exact orientation of the bevel opening.

FIG. 7 illustrates the above described procedure, wherein the tip of the needle is inserted approximately three millimeters laterally to the sagittal plane of the lateral limbus at the level of the superior orbital rim, wherein the practitioner orients the needle upwardly towards the roof of the orbital cavity, preferably with a medial inclination of five degrees. Thereafter, as stated above, the tip of the needle “walks” along the bone of the orbital roof in a curvilinear fashion with the bevel opening facing the globe, until the tip of the needle is at a depth of twenty five millimeters.

Prior to setting forth the invention, it may be helpful to an understanding thereof to set forth definitions of certain terms to be used hereinafter, specifically in relation to present art needles and usage, including hypodermic needles.

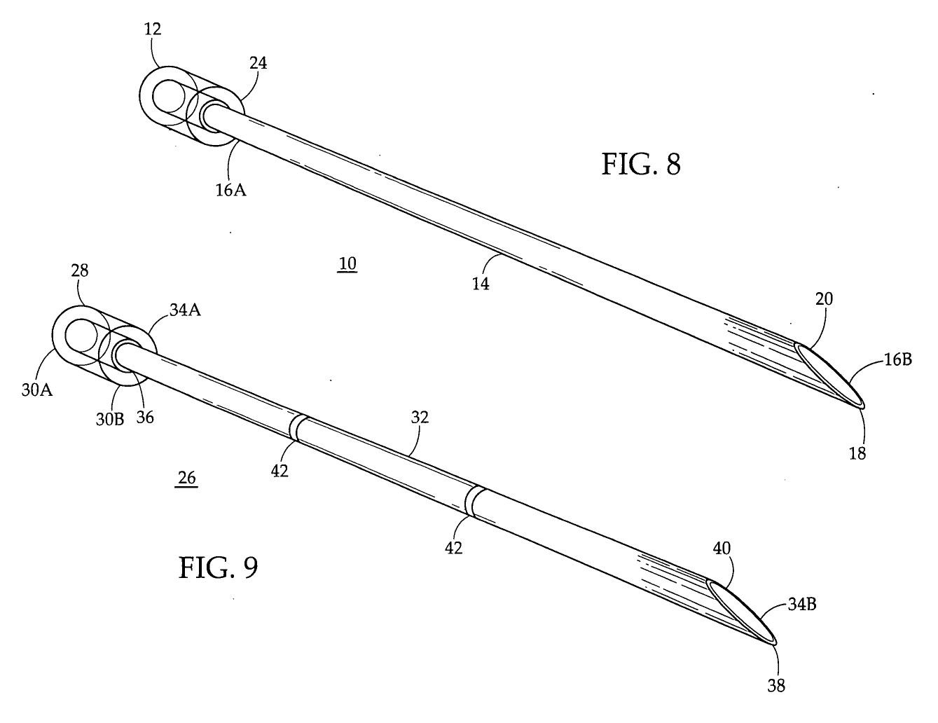

FIG. 8 illustrates a prior art needle 10, wherein the needle is divided into two separate portions, comprising a hub 12 and a shaft 14. The shaft 14 includes a first end 16A and a second end 16B, wherein a tip 18 is locatable at the second end 16B, such that the tip 18 of the needle 10 assists a practitioner in piercing an individual's tissue during a procedure. The shaft 14 also includes a bevel 20 that extends upwardly from the tip 18 of the needle 10 and into the shaft 14. Preferably the shaft 14 consists of a metallic composite, however alternative materials may be used in the production of the shaft 14, including, but not limited to ceramic and plastic. Two types of bevels 20 are currently available in prior art needles 10, sharp and blunt. In most constructions of needles, when a tube of stainless steel is ground to make a point, the more shallow the angle of grind, the less force required for the needle point to penetrate the tissues; the longer the point, the easier to make a sharp point. Currently, the longest bevels utilized most frequently possess a twelve degree angle of grind, however needles may be ground as much as forty-five degrees for a shorter beveled needle. Presently, practitioners who perform ophthalmic anesthesia commonly utilized a needle with a twenty two degree angle of grind. The hub 12 preferably is comprised of a clear plastic material and is attachable to a syringe barrel by a variety of commonly known attachment means in the art, including but not limited to press-fit and twist-on. Furthermore, the hub 12 is preferably available in different colors, wherein distinct colors identify the gauge (i.e. internal diameter) of the needle. Furthermore, a hub junction 24, which consists of the weld between the hub 12 and shaft 14 of the needle 10, enables connection of both portions. Furthermore, two needle lengths are most commonly utilized for ophthalmic regional anesthesia, twenty-five millimeter and thirty-two millimeter.

FIG. 9 illustrates the instant invention wherein a needle 26 comprises two distinct sections, consisting of a hub 28 having a first end 30A and a second end 30B, and a shaft 32, having a first end 34A and a second end 34B. The hub 28 and shaft 32 preferably are joined by an attachment means 36; in the preferred embodiment, the second end 30B of the hub 28 is attachable to the first end 34A of the shaft 32 by the attachment means 36. In one embodiment, the attachment means 36 is a solder joint. The shaft 32 further includes a tip 38 located at the second end 34B and a bevel 40, wherein the bevel 40 extends upwardly into the shaft 14, such that the tip 38 and bevel 40 work in conjunction to penetrate an individual's tissues during a procedure, preferably for the delivery of ophthalmic regional block anesthesia.

The instant invention further includes a plurality of markings 42 to provide a practitioner with an exact measurement of the depth of the needle 26 during a procedure. The markings 42 eliminate any and all guesswork currently employed by a practitioner once the tip 38 and bevel 40 of the needle are no longer visible after insertion, while also creating several reference points allowing visual confirmation of the location of the needle 26. Preferably the markings 42 are equidistantly disposed along the shaft 32 such that the first marking indicates the distance traveled from the tip 38 of the needle to the first marking. Subsequent markings likewise, provide information on the distance of the needle 26 from the tip 38 to each marking 42 along the shaft. It should be noted, that in practice, two lengths of needles are most commonly employed during delivery of ophthalmic regional anesthesia, the first being twenty-five millimeters, and the second being a thirty-two millimeter needle, however the instant invention is applicable to all other lengths, designs, makes and models of delivery that are able to successfully deliver ophthalmic regional anesthesia. Furthermore, the distance at which the plurality of markings 42 are disposed along the shaft vary depending on the individual practitioner, patient, or procedure being performed, and as such, although the preferred embodiment is described below, the needle 26 may incorporate any number of arbitrary distances between markings. In the preferred embodiment, the instant invention will utilize a twenty-five millimeter and a thirty-two millimeter needle 26, wherein each needle 26 includes the markings 42 disposed along the shaft 32 at increments of six and one-quarter millimeters. As such, each needle 26 includes a first marking locatable six and one quarter millimeters from the tip 38, a second marking locatable twelve and one quarter millimeters from the tip 38 and a third marking locatable eighteen and three-quarter millimeters from the tip 38; in this embodiment, the thirty-two millimeter needle 26 also includes a fourth marking locatable twenty-five millimeters from the tip 38. Therefore, a practitioner utilizing the instant invention, and more specifically the preferred embodiment, is able to determine the precise distance the needle 26 has traveled, including when to adjust the angle as described above; further examples of the instant invention are provided below to illustrate the utility in providing for the markings 42. Lastly, it should be noted that each of the markings in the preferred embodiment includes a tolerance of plus or minus one half millimeter, for example the first marking in the preferred embodiment would measure distances from five and three-quarter millimeters to six and three-quarter millimeters from the tip 38. As with other aspects of the instant invention, it is understood in alternate embodiments, that various other tolerances are incorporated depending on the usage etc. of the needle 26.

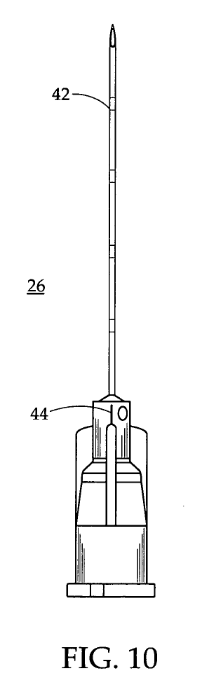

FIG. 10 illustrates one alternate embodiment of the instant invention, wherein the needle 26 includes a linear demarcation 44 to provide a practitioner with the exact orientation of the tip 38 and bevel 40, once the needle 26 has entered an individual's tissues. Preferably the linear demarcation 44 is locatable on the hub 28, such that the demarcation extends downwardly from the first end 30A of the hub 28 to the second end 30B of the hub 28. Alternatively, the linear demarcation 44 is locatable on the shaft 32, such that the demarcation extends downwardly from the first end 34A to the second end 34B of the shaft 32. However, in yet another embodiment, the instant invention may consist of a variety of combinations of markings 42 and the linear demarcation 44, wherein one embodiment consists of only the markings 42, one embodiment consists of only the demarcation 44, and one embodiment includes the markings 42 and demarcation 44.

Now that the invention has been set forth, an explanation of the use of the instant invention with the four previously described techniques will be put forth, along with further discussion of the advantages of the instant invention.

### EXAMPLE 1

### Harvey's Technique

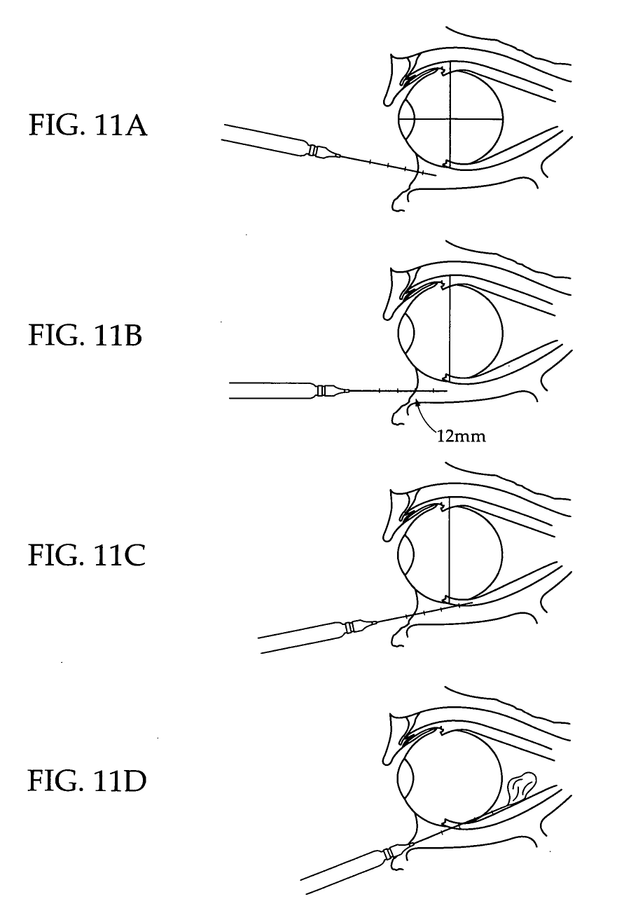

FIGS. 11A-D illustrates use of the instant invention having markings 42 disposed at six and one quarter millimeters intervals along the shaft, and an individual having the cornea right above the orbital rim, and wherein the AL of the globe is twenty-four millimeters. The equatorial plane is therefore twelve millimeters behind the cornea and orbital rim. Therefore when the second marking (i.e. twelve and one half millimeters) (±one half millimeter tolerance) locatable on the instant invention is at the point of insertion, a practitioner knows that the bevel of the needle is at the equatorial plane and that the needle can be redirected per earlier description.

The significant difference between the technique utilizing the instant invention, and the current use of unmarked needles is objectivity, in that it gives the practitioner the exact location of the bevel/tip and allows the practitioner to make the necessary upward correction that with a decreased risk of eye perforation.

### EXAMPLE 2

### Harvey's Technique

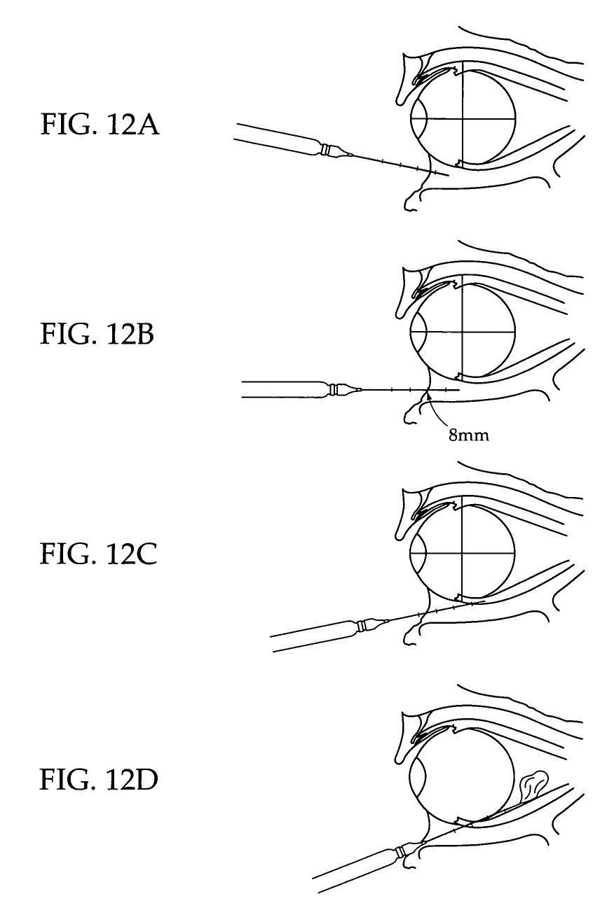

FIGS. 12A-D illustrate use of the instant invention having markings 42 disposed at eight millimeters intervals along the shaft, with an individual having the same twenty-four millimeter AL with an equatorial plane at twelve millimeters, but the cornea is four millimeters in front of the orbital rim, (i.e. the insertion point). The tip of the needle has already passed four millimeters when it is inserted, and only has eight millimeters more to advance before being at the level of the equatorial plane.

Thus a practitioner must consider the redirection change at the eight millimeters mark of the needle, as opposed to the twelve millimeter mark in Example 1. Again, the instant invention will show exactly when the eight millimeters mark is at the level of the initial insertion and consequently, the tip has passed the equatorial plane, thereby allowing the practitioner to make an upward correction.

### EXAMPLE 3

### Harvey's Technique

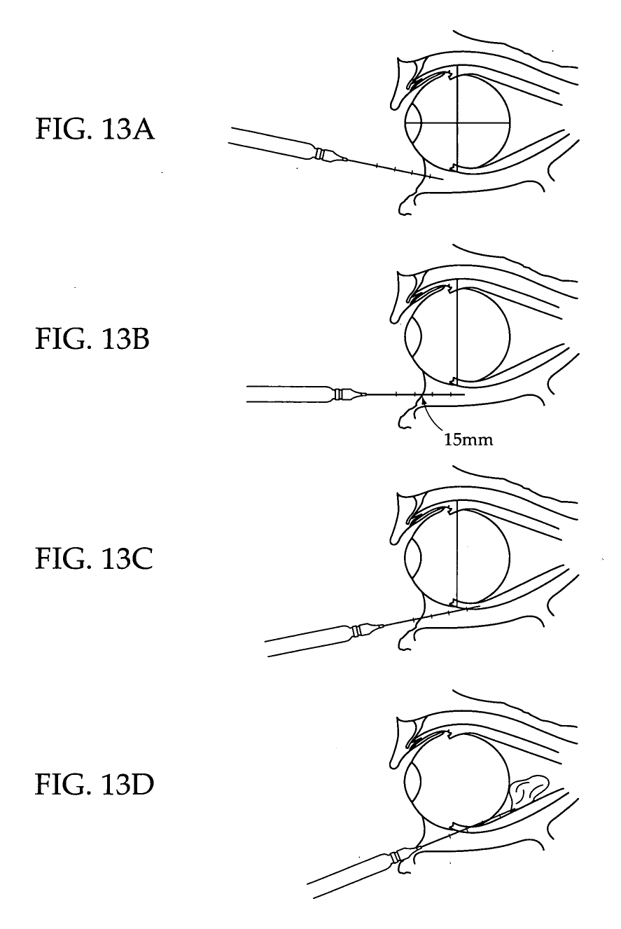

FIGS. 13A-D illustrates use of the instant invention having markings 42 disposed at seven and one-half millimeter intervals along the shaft, and an individual with a long eye with an AL of thirty millimeters and an equatorial plane locatable at fifteen millimeters. In this example, the long eye is less common than the average twenty-three to twenty-four millimeter eye, and has an increased risk for perforation due to a change in direction of the tip taken too early.

For this example, one must assume that the cornea is three millimeters in front of the orbital rim. As such, a practitioner will know precisely when the tip of the needle will be at the equatorial plane when the fifteen millimeter mark is at the point of insertion, thereby requiring an upward angle change. The unique safety element of the instant invention in providing an exact measurement of the depth of the needle is extremely important especially in this type of long AL eye, because there is a higher risk of perforation of the globe.

### EXAMPLE 4

### Hamilton's Technique

### Inferotemporal Retrobulbar Block

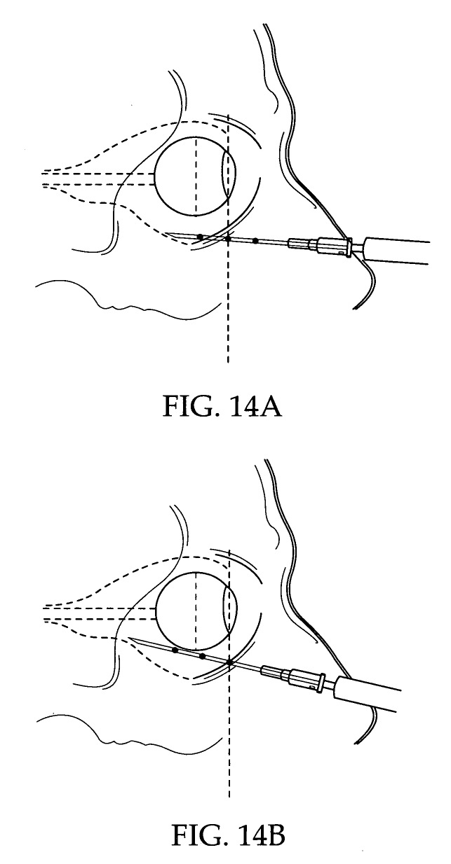

FIGS. 14A-B illustrates the use of the instant invention having markings 42 disposed at six and one-quarter millimeter intervals along the shaft. Therefore, when performing this procedure, the practitioner will know when half of the needle (i.e. twelve and a half millimeters if using a twenty five millimeter needle) is at the plane of the iris, thereby conferring that the tip 38 of the needle has passed by the equatorial plane, and the medial and upward correction is necessary. As stated in previous examples, the identification of when half of the needle is at the level of the iris is crucial, based on the necessary redirection; prior art needles cause the practitioner to estimate the depth of insertion. Therefore, when performing this block using the instant invention, the professional will know precisely when half the needle has reached the plane of the iris and must be redirected in order to position the tip of the needle five to seven millimeters behind the globe.

Thus, once the needle is redirected medially and upward, the advancement of the needle must be stopped, when the twenty-five millimeter mark reaches the plane of the iris.

Another important element to consider while performing this procedure is the orientation of the bevel of the needle, such that it is commonly accepted that the bevel face the globe during insertion and withdrawal of the needle. However, when a practitioner uses a prior art, it is extremely difficult for the practitioner to know the orientation once the tip of the needle has been inserted and is inside the tissues surrounding the eye. It should be noted that “orientation” of the bevel, refers to which side (superior, inferior, right or left) the bevel of the needle is facing.

FIG. 14A illustrates when half of the needle is at the level of the iris, as indicated by the specific marking, which provides an exact measurement of the depth of insertion prior to the medial and upward redirection. As stated earlier, in this example a twenty-five millimeter needle is employed, such that the twelve and one-half millimeter marking indicates that correction must occur.

FIG. 14B illustrates when the tip of the needle is in the final position behind the eye (final position). In this example a thirty-two millimeter needle is employed and the twenty-five millimeter mark at the level of the iris provides a clear reference as when to cease advancement of the needle.

### EXAMPLE 5

### Medial Compartment and Superotemporal Blocks

As previously discussed during the description of Hamilton's technique, along with the advantages of using the instant invention, the same principles apply to both medial compartment and superotemporal blocks. Regardless of the technique employed, or the depth of insertion of the needle, the instant invention provides the professional a simple method of knowing: 1) where the tip of the needle is located and its relation with the axial length and equatorial plane; 2) when to stop the advancement of the needle; and 3) the orientation of the bevel.

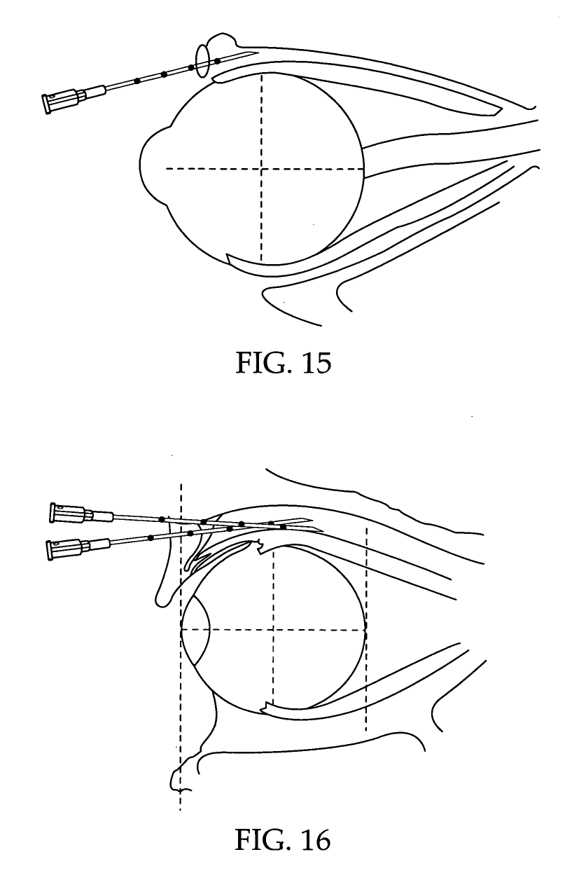

FIG. 15 illustrates the medial compartment block employing a thirty two millimeter needle possessing the linear demarcation 44 on the hub 12. Thus, this embodiment of the instant invention allows for the practitioner to clearly identify the depth of insertion along with providing the orientation of the bevel to the practitioner, thereby knowing the exact side the bevel of the needle is facing.

FIG. 16 illustrates the superotemporal block employing a thirty two millimeter needle that assists the practitioner in safely “walking” the tip of the needle along the bone of the orbital roof in a curvilinear fashion while still knowing the depth of insertion.

### REFERENCES

Ophthalmic Anesthesia
Chandra Kumar, Chris Dodds, Gary Fanning
Ophthalmic Anesthesia
G Barry Smith, Robert A. Hamilton, Caroline A. Carr
Opthalmic Anesthesia
James P. Gills, Robert F. Hustead, Donald R. Sanders
Atlas of Clinical and Surgical Orbital Anatomy
Jonathan J. Dutton
Opthalmic Anesthesia Society
20^{th} Annual Scientific Meeting, Oct. 13-15, 2006
Ophthalmic Block Workshop
Northwest Anesthesia Seminars, Inc., Apr. 21-22, 2007
Review of Ophthalmology
http://www.revophth.com/index.asp?page=1_{—}907.htm
Journal of Cataract & Refractive Surgery
Volume 32, Issue 9, September 2006, pages 1401-1402

What is claimed is:

1. An insertion mechanism for use with a syringe comprising:
a hub, wherein the hub includes a first end and a second end;
a shaft, wherein the shaft includes a first end and a second end;
a tip, wherein the tip is locatable at the first end of the shaft for insertion into an individual during a procedure;
a bevel, wherein the bevel extends upwardly into the first end of the shaft from the tip;
an attachment means, wherein the attachment means enables joining of the hub to the shaft; and
a plurality of markings equidistantly disposed along the shaft.

2. The insertion mechanism of claim 1, wherein the attachment means is a solder joint.

3. The insertion mechanism of claim 2, wherein the hub is removeably attachable to a syringe by a fitting, wherein the fitting is selected from the group consisting of a press-fit fitting, twist-on fitting, swage fitting and a quick disconnect fitting.

4. The insertion mechanism of claim 3, wherein the plurality of markings disposed along the shaft provide the distance from the tip of the needle to the individual marking.

5. The insertion mechanism of claim 4, wherein the length of the shaft is twenty-five millimeters.

6. The insertion mechanism of claim 5, wherein the plurality of markings are equidistantly disposed along the shaft at six and one-quarter millimeter intervals.

7. The insertion mechanism of claim 4, wherein the length of the shaft is thirty-two millimeters.

8. The insertion mechanism of claim 7, wherein the plurality of markings are equidistantly disposed along the shaft at six and one-quarter millimeter intervals.

9. The insertion mechanism of claim 3, wherein a linear demarcation is locatable on the hub and extends downwardly from the first end of the hub to the second end of the hub to provide the orientation of the bevel during a procedure.

10. The insertion mechanism of claim 3, wherein a linear demarcation is locatable on the shaft and extends downwardly from the second end of the shaft to the first end of the shaft to provide the orientation of the bevel during a procedure.

11. The insertion mechanism of claim 9, wherein the plurality of markings disposed along the shaft provide the distance from the tip of the needle to the individual marking.

12. The insertion mechanism of claim 10, wherein the plurality of markings disposed along the shaft provide the distance from the tip of the needle to the individual marking.

13. The insertion mechanism of claim 11, wherein the plurality of markings are equidistantly disposed along the shaft at six and one-quarter millimeter intervals.

14. The insertion mechanism of claim 12, wherein the plurality of markings are equidistantly disposed along the shaft at six and one-quarter millimeter intervals.

15. The insertion mechanism of claim 11 or 12, wherein the insertion mechanism is utilized for the delivery of ophthalmic regional anesthesia.

16. The insertion mechanism of claim 15, wherein the insertion mechanism is utilized in the performance of a medical procedure selected from the group consisting of an inferotemporal retrobulbar/peribulbar blocks, medial compartment peribulbar block and a superotemporal peribulbar block.
